(19) United States
(12) Patent Application Publication
(10) Pub. No.: US 2003/0040784 A1
(43) Pub. Date: Feb. 27, 2003

(54) Medical lead adapter for storing, isolating, identifying, and connecting temporary pacing leads

(76) Inventors: Scott Vincent Pasternak, Springfield, MO (US); Frank Pascoe Davis, Springfield, MO (US)

Correspondence Address: Mr. Scott V. Pasternak, 218 E. Norton Rd Apt 10, Springfield, MO, 65803, US

(21) Appl. No.: 10/219,186

(22) Filed: Aug. 15, 2002

Related U.S. Application Data
(60) Provisional application No. 60/313,706, filed on Aug. 21, 2001.

(51) Int. Cl. A61N 1/372

(52) U.S. Cl. 607/116

(57) ABSTRACT

A medical lead adapter that provides for the easy storage, electrical isolation, accurate identification, and quick connection to an external temporary cardiac stimulator or pacemaker in patients who have undergone temporary pacing wire implantation. The invention facilitates making multiple, secure connections simultaneously, thus alleviating any delay in pacemaker connection. In its preferred embodiment, the device will accommodate pacing leads from multiple manufacturers as well as provide a configurable receptacle to interface with any pacemaker or cardiac stimulator.

### CROSS REFERENCE TO RELATED APPLICATION

This application claims priority from U.S. Provisional Application No. 60/313,706, filed Aug. 21, 2001, entitled “MEDICAL LEAD ADAPTER FOR STORING, ISOLATING, IDENTIFYING, AND CONNECTING TEMPORARY PACING LEADS”, and is fully incorporated herein by reference.

### STATEMENT REGARDING FEDERALLY SPONSORED RESEARCH OR DEVELOPMENT

Not Applicable.

### BACKGROUND OF THE INVENTION

The present invention generally relates to an adapter to semi-permanently store, isolate, identify, and rapidly connect temporary pacing wires to an external cardiac stimulator or pacemaker in patients who have undergone temporary pacing wire implantation for a variety of reasons, including cardiothoracic surgery.

Following cardiothoracic surgery, patients often develop the need for temporary, episodic cardiac pacing. For this reason, the surgeon implants pacing leads that provide a means for stimulating the heart. Often these leads consist of two unipolar ventricular leads and/or two unipolar atrial leads. The implanted ends of these leads lie epicardially or while the externalized ends are brought through the chest wall, secured, and made available for either connecting to an external cardiac stimulator or storage in an electrically isolated manner. The bodies of these leads are insulated while the externalized ends typically are made of a stainless steel or other metal pin that is suitable for connection to the terminals of a pacemaker or pulse generator either directly or by means of a cable supplied by the pacemaker manufacturer.

When these wires are not connected to the cable or pacemaker, they are to be stored in an electrically isolated manner in order to prevent microshock electrocution, a phenomenon where the pacing lead may come into contact with small amounts of electric current, as in static electricity for example. This is often accomplished by placing the wires into syringes and taping these syringes to the chest wall. This assumed electrical isolation often fails because the syringe becomes dislodged on the patient gown or the connecting pins on the leads migrate out of the hole at the end of the syringe. It is also important to note that these wires are often unlabeled and are only identifiable by knowledge of the implanting physician's usual placement of the leads (for example, atrial on the right and ventricular on the left).

In the current state of technology, when the need arises to institute cardiac stimulation or pacing, the clinician must locate the syringes, which often become taped into the chest dressings found on cardiothoracic surgery patients. The tape must be removed from up to two syringes, the wires unraveled from the syringe barrels, and up to four connections made to the pulse generator while maintaining electrical isolation prior to the initiation of pacing. These connections are made by inserting the metal connecting pins into the pacemaker connector, one at a time, and either tightening screws to make each connection or using quick connect squeeze-and-release devices. During all of this, the clinician must be certain to properly identify and connect the atrial and ventricular wires to their respective places on the patient cable and then connect the cables to the proper place on the pacemaker or pulse generator. Mistakes in this step may lead to reversed atrioventricular pacing or the delivery of rapid atrial pacing to the ventricles.

It has been observed that this complex series of connections during periods when the clinician experiences increased stress (when patients develop absolute or relative bradyarythmias, loss of atrioventricular conduction and synchrony, or asystole, as examples) sometimes fails to be completed successfully, the most common problems being forgetting to tighten the screws on the pacemaker cable which results in intermittent pacing and sensing and failing to connect the wires to the proper locations on either the cables or the pacemaker itself. It has been also observed that this leads to a significant delay in the initiation of pacing. Frequently observed is the common complaint that “the pacemaker wires don't work” and frequently this situation is rectified by re-verifying all of the steps necessary to make the connections.

In our present invention, we have solved all of the aforementioned disadvantages of the current technology and art by utilizing that which is described below.

### BRIEF SUMMARY OF THE INVENTION

It is an object of our present invention to solve the needs identified in the background material provided. In its preferred embodiment our invention consists of plug which resides on the patient, housing, storing, identifying and electrically isolating the pacing leads. The invention also includes a receptacle that connects to the pacemaker and receives the aforementioned plug. During lead implantation or thereafter, the pacing leads are placed into the plug in their labeled locations and the plug is closed. The plug, once housing and storing the leads provides for organizing, identifying and affording quick connection (via plugging the plug into the receptacle) to the pacemaker or pulse generator. The storage of the leads in the plug assembly assures electrical isolation and accurate identification. This plug assembly could be taped to the patient's chest wall. Connection of the pacing leads to the pacemaker by plugging the plug into the receptacle is a one step process that would allow quick, secure, isolated and accurate connections of up to four leads simultaneously. The ease of accurately connecting all wires at once would decrease the time to pacing initiation as well as decrease the stress that the staff experiences trying to make multiple connections.

Our present invention also allows for optional quick access to the pacing leads for performing unipolar or bipolar atrial electrocardiograms. Simply probing the atrial wires through the access holes provided on the plug assembly would allow one to view the patient's atrial rhythm on the electrocardiogram monitor.

Our present invention allows access to the metal pins that terminate the pacing leads in case of suspected device failure or incompatibility with the available connections by opening the plug assembly and removing the pins from their labeled chambers.

Our present invention also allows for the use of multiple types of pacing leads from a variety of manufactures. The holder inside the plug that engages the metal pin of the pacing lead will accept leads having various outer diameters. Also, the invention allows for connections to multiple types of pacemakers by modification of the cable leading from the receptacle to the pacemaker. Alternate embodiments would include a means to use the metal pins on the pacing leads themselves to make the connection to the receptacle, though this would decrease the ability to use varieties of pacing leads from multiple manufactures.

With these aforementioned improvements, our invention solves the problems of storage and isolation, identification, and simplifies the work of making the connections accurately, thus reducing the time from the decision to start pacing to actual pacing.

### BRIEF DESCRIPTION OF SEVERAL VIEWS OF THE DRAWING

FIG. 1 depicts a top-down view of the plug assembly, opened, with a detailed view of the interior layout of the plug assembly in its preferred embodiment.

FIG. 2 depicts a frontal three dimensional view of the plug assembly, opened, with a detailed view of the layout of the front of the plug assembly.

FIG. 3 depicts a frontal three dimensional view of the plug assembly, closed, embodied without the optional ports for atrial electrocardiograms.

FIG. 4 depicts the a frontal three dimensional view of the receptacle assembly that mates with the plug assembly.

### DETAILED DESCRIPTION OF THE INVENTION

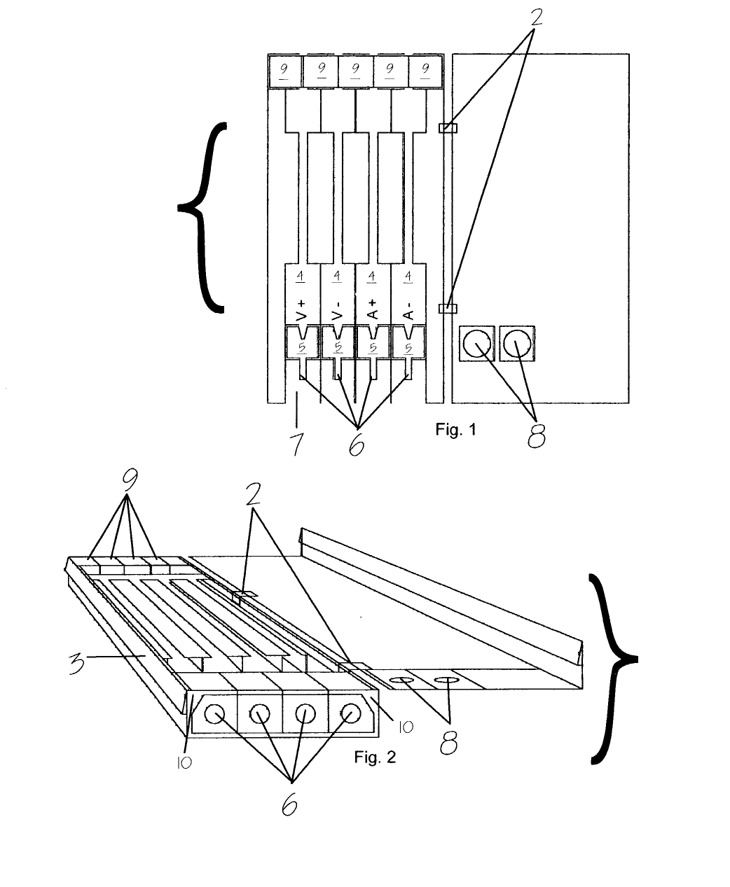

The invention, in its preferred embodiment consists of two parts, a plug (FIGS. 1 thru 3) which remains attached to the patient and a receptacle (FIG. 4) that is attached to the cable leading to the pacemaker.

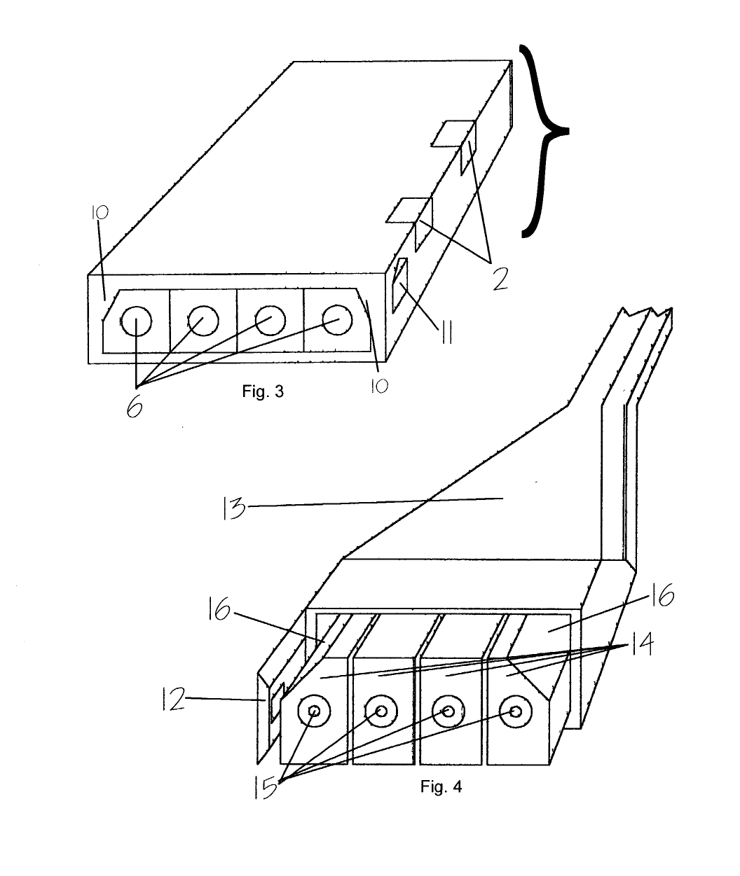

The plug assembly consists of a plastic case (1) that is hinged (2) on one side and is closeable by means of a clip (3) closure on the opposing side. The clip closure (3) is designed such that in the event of device failure, the case may be opened by bending back or breaking the clip off, thus exposing the labeled metal connecting pins (not shown) of the pacing leads (not shown) themselves. There are separate chambers (4) inside of the plug to accommodate the metal connecting pins on the pacing wires. In each chamber is a metal electrical connector (5) that receives the connecting pin by means of an interference fit and is connected to the electrode (6) on the front (FIGS. 2 and 3) of the plug assembly which is recessed (7) to prevent accidental microshock electrocution or contamination. There is a cover (not shown) that is applied to the front (FIGS. 2 and 3) of the assembly to further isolate the pacing electrodes (not shown) when they are not in use. Towards the back of each chamber there is a means for securing the pacing wire (9) to prevent accidentally pulling the wire out of the electrical connector (5). The use of this electrical connector (5) allows for the accommodation of different types of pacing wires with different outer diameters. On top of the atrial chambers there are optional access holes (8) for obtaining atrial electrocardiograms. Within each chamber there is a label (identified in FIG. 1 as A+,A−,V+,V+) identifying to the surgeon or clinician installing the lead and to the clinician who opens the case in the event of suspected failure the anatomical location of each lead. The outer body of the plug case (seen well in FIGS. 2 and 3) contains directional angles (10) that ensure unidirectional matching with the receptacle to make certain that there is no possibility to plug the plug assembly backwards into the receptacle (FIG. 4). The outer body of the plug case also has a notch (11) for snapping the plug into the receptacle and engaging the notch receiver (12).

The receptacle assembly (FIG. 4) consists of a plastic case (13) that at it's front has extended plugs (14) to reach into the front of the plug assembly (FIGS. 2 and 3) which has been recessed (7) to protect the electrodes (6). In each of these plugs (14) there is an electrical connector (15) that engages the pacing electrode (6) found on the front of the plug assembly. The remainder of the inside of the receptacle assembly is designed to allow the interfacing of the pacemaker cable (not shown) to the electrical connectors (15). This could also be accomplished, of course, in the part of the cable that connects to the pacemaker (not shown), allowing standardized production of the receptacle (FIG. 4). The outer body of the receptacle contains a notch receiver (12) that engage the notch (11) of the plug in order to hold the two parts (FIGS. 1 thru 3 collectively as the plug assembly to the receptacle assembly in FIG. 4) of the invention snugly together. The outer body also contains directional angles (16) to ensure the unidirectional matching with the plug assembly to make certain that there is no possibility reverse the connection and connect the plug backwards.

The above description is illustrative and not restrictive. Many variations of the invention will become apparent to those of skill in the art upon review of this disclosure. The scope of the invention should, therefore, be determined not with reference to the above description, but instead should be determined with reference to the appended claims along with their full scope of equivalents.

We claim:

1. An adapter assembly for externalized temporary pacing leads which is comprised of two members:
a plug member made from non-conductive, non-corrosive, non-porous material which resides externally on the patient, providing a means of storage, electrical isolation, identification, and providing an electrical connection between the pulse generator or pacemaker and said pacing leads;
a receiver member made of similar material to provide a means of universal connection between said plug and pulse generator or pacemaker.

2. The body of the plug member as defined in claim 1 is comprised of:
a rigid body of the material in claim 1;
a hinge on one side;
a safety latch on an opposing side which when closed creates a fluid-tight seal which can be reopened by practitioners in case of emergency;
a recessed, sealed electrical connection to engage the receiver member defined in claim 1 on a third side;
a sealed entrance into the internal chamber through which said leads would enter;
a shape which would make it impossible to plug into other healthcare devices or the pulse generator/pacemaker in an improper fashion.

3. The internal chamber of the plug member as defined in claim 2 is:
a sealed chamber to receive the ends of said leads through a water-resistant seal into a space where excess leads can be electrically isolated, stored, and connected to electrical connectors or be mounted so as to become the electrical connection themselves;
a means of identification of the leads by labels applied to the plug body.

4. The closable portion of the plug defined in claim 2 contains:
a fluid-resistant, resealable interface positioned above the atrial electrical connections affording quick access for unipolar or bipolar atrial electrocardiograms.

5. The receiver member in claim 1 contains:
a space for electrically connecting the plug defined in claim 1 with any pacemaker or pulse generator in a manner that ensures proper signal distribution to appropriate leads when the two members are properly connected together;
a means of securing the two members together securely in a unidirectional manner, preventing accidental reversal of electrical connections.
